(19) United States
(12) Patent Application Publication
(10) Pub. No.: US 2004/0072226 A1
(43) Pub. Date: Apr. 15, 2004

(54) Movement of multi-enzymatic nanoassemblies on recognition landscapes

(76) Inventor: Milan N. Stojanovic, Fort Lee, NJ (US)

Correspondence Address: Cooper & Dunham LLP, 1185 Avenue of the Americas, New York, NY, 10036, US

(21) Appl. No.: 10/613,363

(22) Filed: Jul. 3, 2003

Related U.S. Application Data
(60) Provisional application No. 60/393,691, filed on Jul. 3, 2002.

(51) Int. Cl. C12Q 1/68; C07H 21/02

(52) U.S. Cl. 435/6; 536/23.1

(57) ABSTRACT

A macromolecular assembly has a body and at least four catalytic leg units having nucleic acids and is adapted to travel across a layer of oligonucleotide fuel substrate molecules by having each leg unit recognize and bind to a fuel substrate, cleave the fuel substrate and search for a new fuel substrate, said leg units alternately binding and cleaving out of phase to keep at least one leg unit bound to a fuel substrate. The fuel molecules may be arranged to provide a gradient, whereby the assembly travels along the gradient.

### CROSS REFERENCE TO RELATED APPLICATION

This application claims priority on U.S. Ser. No. 60/393,691 filed Jul. 3, 2002, which is incorporated by reference herein.

### BACKGROUND OF THE INVENTION

The present invention relates to multi-enzymatic nano-assembly structures that move directionally on recognition landscapes or substrates.

Throughout this application, various publications are referenced to as footnotes or within parentheses. Disclosures of these publications in their entireties are hereby incorporated by reference into this application to more fully describe the state of the art to which this invention pertains. Full bibliographic citations for these references may be found at the end of this application, preceding the claims.

Despite impressive progress (1-6) in the engineering of artificial and biological motors, important difficulties remain unresolved. In contrast to biological motors, all artificial molecular motors are unable to undergo continuous conformational changes in the presence of an excess of fuel; instead they require intervention by an operator (i.e. via the addition of anti-fuel) to complete the cycle and prepare for the next movement. Also, efforts to spatially direct movements of individual motors and to operate multiple motors coherently in order to achieve macroscopic effects, i.e. directed movement of objects on nanoscale or mesoscale, has not been successful. Further, because biological motors are optimized by evolution to function in specific environments, incorporating them into new systems requires extraordinary amounts of retro- and forward engineering.

### SUMMARY OF THE INVENTION

The present invention takes a novel approach, one that does not use known artificial or natural motors. The present invention provides de novo molecular machines, and defines rules that describe their behavior. The invention can be used for applications in which these rules are not restrictive. They can be constructed using nucleic acid-based sensors, catalysts and computation elements (7-11).

The present invention provides a new paradigm in the construction of artificial molecular scale machines. Unlike all previous models, continuous movement is achieved through coupling the catalytic reactions of oligonucleotides to their interactions with recognition landscapes, and spatially directed movement is achieved through a gradient in the density of substrate-fuel in the recognition landscape, or a chemical gradient in solution, or an asymmetry among the subunits of the machine.

The present invention provides molecular “spiders”, which travel along a feed substrate by catalytic reactions. The substrate consists of tethered DNA/RNA oligonucleotide “fuel” molecules. The spider design may comprise streptavidin complexed with four or more oligonucleotide-based (deoxyribozyme) catalyst “legs.” The spider moves along the substrate in search of fresh fuel leaving behind a trail of exhausted fuel. Each leg experiences a fuel consumption cycle: recognition, fuel cleavage, and product release. The plurality of legs enables the spider to achieve movement on the substrate surface after one leg achieves cleavage, it is able to seek fresh a fresh fuel site, while other legs are still bound by recognition. Thus, the legs move out of phase. The tethered product fragments can be labeled (flourescently or with gold nanoparticles) to investigate the trail of movement.

The invention provides programmable molecular scale machines that would move directionally for as long as they are provided with fuel. These machines would require no intervention by an operator beyond an initial instruction set given in the form of a recognition landscape.

A nanoassembly with a plurality of, e.g., four or more deoxyribozymes (“molecular spider”) would move on surfaces with attached substrate-fuels. The nano-assemblies may be constructed from inert carriers and deoxyribozymes with phosphodiesterase activity. The substrate-fuel may be hybrid DNA/RNA, and may be arranged in a gradient to define a movement path for the nano-assemblies.

According to the present invention, a macromolecular assembly is provided comprising a body and at least four catalytic leg units having nucleic acids, the assembly being adapted to travel across a layer of feed oligonucleotide fuel substrate molecules wherein each catalyic leg unit recognizes and binds to a fuel substrate, cleaves the fuel substrate and searches for a new fuel substrate, said leg units alternately binding and cleaving out of phase to keep at least one leg unit bound to a fuel substrate.

According to another aspect of the present invention, a macromolecular system is provided, comprising a macromolecule as described above, and a feed layer having oligonucleotide substrates as fuel molecules.

### DESCRIPTION OF THE DRAWINGS

FIG. 1—Shows movement of a four-legged spider nanoassembly on a surface with tethered substrate-fuel (side view): A→B. Spider attaches to surface with multivalent binding to the surface; B→C. Enzymatic leg-1 cleaves substrate (B) and C→D “searches” for another substrate (D), while another substrate gets cleaved (D, leg-3); D→E. Leg-1 binds a new substrate, while leg-3 searches for a substrate and yet another gets cleaved (leg-4); In D→E spider's center of gravity moved toward leg-1; F. Over time spider moves, while irreversibly changing the surface. Legs could be all equivalent or could be different to form asymmetric spiders.

FIG. 2—A: Shows cleavage of hybrid substrate by reaction of deoxyribozyme 12E (13) modified with biotin; fluorogenic reaction is shown with substrate double end-labeled. Upon cleavage there is an increase in fluorescence emission of fluorescein F, as black hole BH quencher is removed. When substrates are attached to surfaces, F is substituted with amino group and there is no BH. Two other deoxyribozymes 10-23 (14, substrate changes to central 5′ rGrU) and 17E (15) may also be used. B: Streptavidine complexed with four biotin-labeled deoxyribozymes grabbing and cleaving the fuel in solution. Streptavidine is a tetramer organized in D2 point symmetry, however with an appropriate linker length this should not be a factor.

FIG. 3: Shows alternative construct having four and six deoxyribozymes attached at double helix or three-way junction. Wiggly line represents flexible polyethylene glycol spacers.

FIG. 4: Shows chemistry of the attachment of oligonucleotides to the slides (cf. text).

### DETAILED DESCRIPTION OF THE INVENTION

According to the present invention, a macromolecular assembly is provided comprising a body and at least four catalytic leg units having nucleic acids, the assembly being adapted to travel across a layer of feed oligonucleotide fuel substrate molecules wherein each catalyic leg unit recognizes and binds to a fuel substrate, cleaves the fuel substrate and searches for a new fuel substrate, said leg units alternately binding and cleaving out of phase to keep at least one leg unit bound to a fuel substrate.

The leg units may have the same nucleic acids or may have different nucleic acids. The assembly may have at least six catalytic leg units.

The four leg units may be arranged in a tetrahedral relationship, or may be arranged in a rectangular relationship, for example.

The leg units may be comprised of DNA enzymes, and/or RNA enzymes.

The body may be comprised of streptavidine. The body may be comprised of DNA and/or RNA.

The leg units may include flexible polyethylene glycolspacers.

According to another aspect of the present invention, a macromolecular system is provided, comprising a macromolecule as described above, and a feed layer having oligonucleotide substrates as fuel molecules.

The oligonucleotides may be made of DNA, RNA or a mixture thereof.

The fuel molecules may be arranged in a gradient on the substrate. The leg units may comprise different nucleic acids.

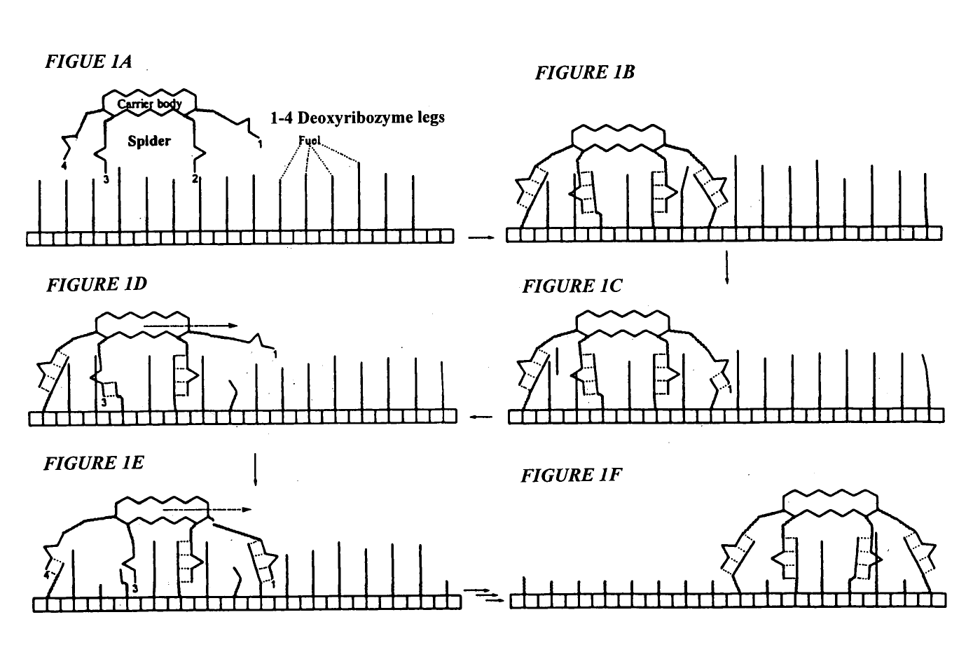

As used herein, the term “molecular spiders” or “spiders” means macromolecules with multiple attached DNA enzymes (deoxyribozymes, 12) that interact with and degrade substrates-fuels (FIG. 1). The spiders attach to surfaces displaying tethered substrate-fuel and move on these surfaces through cycles of fuel recognition (“injection”), fuel cleavage (“burning”), and product release (“exhaust”). The spiders are attached through multivalent binding of enzymes to surfaces. With an appropriately calibrated binding affinity (Km) and turnover rate (^{k}_{cat})spiders are unlikely to be released in solution as long as they can rapidly capture another substrate before all of their enzymatic units are released. The inert machromolecular bodies of spiders can be constructed from various oligomeric proteins, branched DNA molecules, dendrimers, or even smaller polymer particles. The key functional components of molecular spiders are “motor-sensor legs”, oligonucleotide-based catalysts that cleave the substrate-fuel and release waste products. Deoxyribozymes exert phosphodiesterase activity on hybrid DNA/RNA oligonucleotides and spiders may contain four to six identical deoxyribozyme units. Asymmetric spiders may be provided with non-identical legs, as they may leave traces with designer shapes. Chemotaxis of these nanoassemblies may move up and down gradients of fuel on surfaces and gradients of allosteric promoters in solution.

The history of the movement along the fixed gradient can be followed by tracing the tethered fragments of consumed fuel. Consumed fuel can be chemically modified selectively over intact fuel. This modification can be used to direct deposits of metallic materials (e.g. gold nanoparticles) to traces, which are then imaged by scanning electron microscopy, or one can fluorescently label products, and observe shapes of the traces through various fluorescent microscopies.

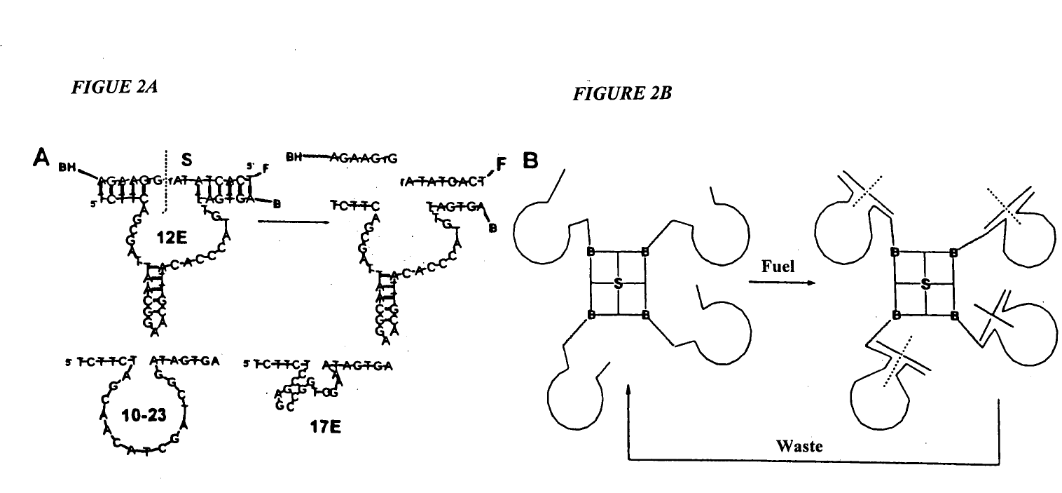

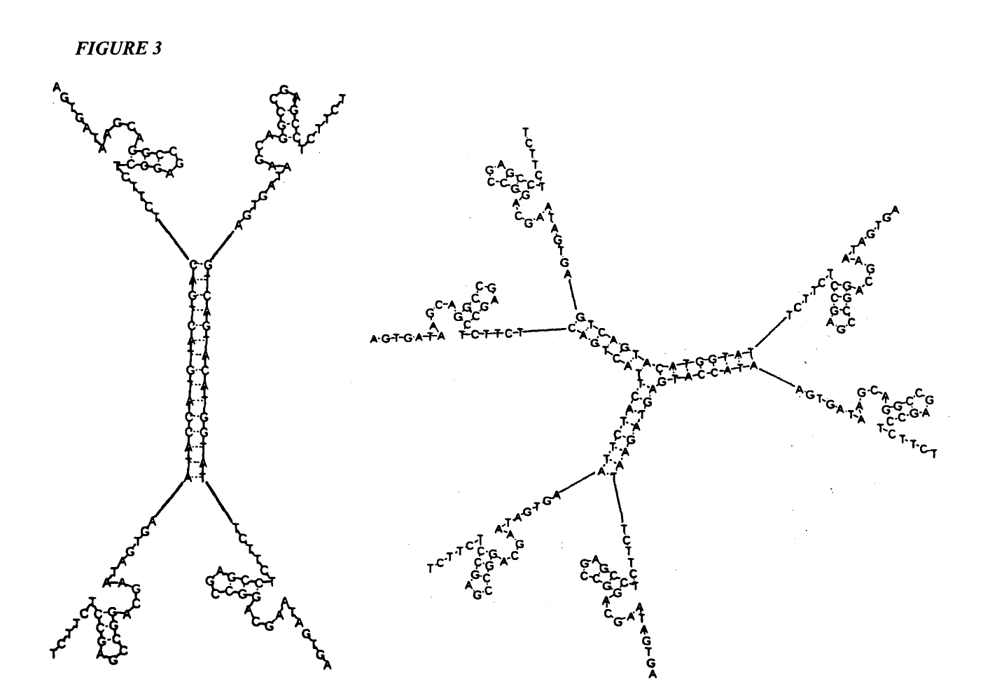

Steptavidine may be employed in a complex with four 3′ biotin-labeled deoxyribozymes (FIG. 2B), four deoxyribozymes attached to a double helix, and six deoxyribozymes attached to a three-way junction (FIG. 3). Oligonucleotides may be custom-made, and no covalent chemistry of attachment will be involved. Components may be mixed together followed by appropriate purification.

Streptavidine and three different deoxyribozymes (13-15) may be used with turnover numbers spanning over three orders of magnitude, from 0.04 to 4 per minute. Long polyethylene glycol (PEG) spacers for 3′ biotin attachment may be used for each deoxyribozyme. One can construct the assemblies in FIG. 3 by mixing individual chains and purifying resulting DNA spiders by gel electrophoresis. One can test whether the assemblies retain the capacity for multiple turnovers in the presence of an excess of substrate, and then identify conditions (concentration of salts and divalent metal ions) that result in the largest spread among cleavage rates. These optimizations may be done in the presence of double end-labeled fuel for fluorogenic detection of cleavage (7). In the optimization process one can test other PEG linker lengths in deoxyribozymes (in order to change the size and flexibility) and fuels with mismatches which may increase the turnover rates, facilitating the release of the products. The all-DNA constructs (FIG. 3) may have their length and flexibility optimized and bends may be introduced through bulges. Four-way junctions may provide eight-legged spiders. Fluorogenic detection may be key for rapid initial characterization of spiders.

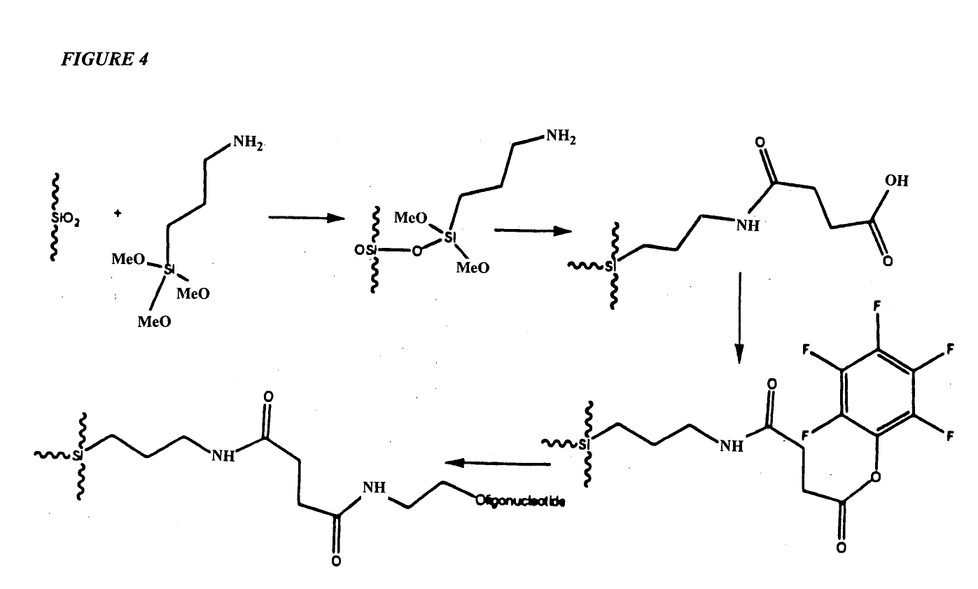

Upon determination that the spiders are indeed catalytically active in solution, one may study their movement on glass slides to which deoxyribozyme substrates have been tethered (FIG. 4). One may initially make glass slides with several different concentrations of substrates, although one can also use silicon chips or gold surfaces, including Biacore chips. One can use published procedures to functionalize glass slides with amino groups through reaction with 3-aminopropyltrimethoxysilane (16-17). One can then react amine-modified glass slides with succinic anhydride. Activation of the resulting acidic functionality to its pentaflurophenol ester will allow coupling to oligonucleotide fuel modified with amines. Concentration of fuel can be controlled by mixing amino-derivatized fuel with irrelevant amines during coupling with tethered pentafluroesters. One can test all spiders on one fuel surface at first. To optimize this surface, one can change various parameters in the fuel, including inclusion of mismatches in the 5′ and 3′ regions, the length of PEG spacers and chemistry of attachment.

Spiders may be applied to derivatized glass slides in buffer that is non-supportive of catalytic reaction (i.e. no bivalent ions), at initial concentrations of ˜1 spider per 100 μm². The slides may be incubated in a buffer facilitating catalysis for various periods of time. One may expect spiders to move in two dimensions on slides, without detaching from the surface, because at any moment the likelihood is that at least one of the deoxyribozymes will be attached to the surface. In the unlikely event that four-legged spiders are released too rapidly from surfaces, one can test deoxyribozymes with longer substrate recognition arms (higher Km) or with more arms. Proper controls would include testing the system with non-cleavable fuel and fuel containing mismatches; testing with inhibitors of catalytic activity and with three-, two- and one-legged spiders. Other interesting experiments that may be performed are studies of the movements of spiders with three functional deoxyribozyme legs, and with four deoxyribozyme legs, but with mixed spacers and mixed cleavage rates. In all these instances, the plaques of the spent fuel will probably on average not point in any direction; however, their dimensions and shapes may depend on types of constructs used. In the experiments with asymmetric spiders initial orientation of spiders on the surface will likely influence the directionality of the traces, and that one can induce interesting shapes in traces (e.g. spirals or curvatures).

In the initial experiments, individual tracks of spiders may be visualized through selective modification of the cleaved fuel. For example, by using fuel with phosphorylated 5′ ends cleavage would specifically create a non-phosphorylated oligonucleotide attached to the substrate surface. Reacting this product selectively with γS-ATP in the presence of T4 polynucleotide kinase would give one reactive thiols at the 5′ end of the tethered products, and would immediately allow one to label the trace with 6-iodoacetamido fluorescein for scanning fluorescence microscopy. Thiols can be also used to deposit gold nanoparticles for scanning electron microscopy. Alternatively, the movements of individual apiders labeled with fluorescein could be visualized with confocal fluorescence microscopy. The fuel attachment can also be reversed to generate phosphomonoesters at the 3′ end of the cleavage products. These can then be slectively functionalized as well, through various couplings to amines. The traces of cleavage products, their shapes and lengths may be used to characterize spiders. From the foregoing description and tests, one can understand the relationship between spider characteristics, fuel concentration and motility, and will be able to more precisely define important parameters for the chemotasis experiments.

One may pursue the chemotaxis of assemblies along gradients of fuel. These experiments may be performed in the same way as those described herein, except one may construct glass slides displaying gradients. One may include demonstrations that other types of recognition can be connected to chemotaxis in gradients of attractants and repellants, such as recognition by aptamers and nucleic acid enzymes of small molecules and short oligonucletides. One may also use surfaces with constant fuel concentrations but with gradients of allosteric promoters in the solution phase. One could thereafter diverge into two areas. In the first area, one could target movement on biomimetic membranes triggered and directed by gradients in solution. In the second area, one could take advantage of the programmable nature of the movement of “spiders” to use them to irreversibly change surfaces in such way as to facilitate the deposition of conducting materials. Envisioned practical applications include nanopatterning, tissue repair, detection of mechanical defects on surfaces and construction of intelligent sensors and drug delivery tools.

Two examples of making the spider assemblies are given below.

### EXAMPLE 1

Streptavidine is mixed with the excess of nucleic acid catalysts containing biotin at 5′ ends. Unreacted catalysts are removed by passing reaction mixture through streptavidine columns.

### EXAMPLE 2

Oligonucleotide A (i.e. half-spider A) contains two nucleic acid catalysts at 5′ and 3′ ends, separated by inert spacers and a central oligonucleotide stretch. Oligonucleotide B (i.e. half-spider B) contains two nucleic acid catalysts at 5′ and 3′ ends, separated by inert spacers and a central oligonucleotide stretch. Two central stretches are complementary. Half-spiders A and B are mixed at equimolar ratios, heated at 95 degrees C. and slowly cooled to room temperature. The complementary stretches combine and this process leads to the formation of a full spider, which could be purified through non-denaturing gel electrophoresis.

Although a preferred embodiment has been shown and described the invention is not limited thereby and its scope is defined in the appended claims.

References

1. Ballardini, R., Balzani, V., Credi, A., Gandolfi, M. T. & Venturi, M. Artificial Molecular-Level Machines: Which Energy To Make Them Work, Acc. Chem. Res. 34, 445-455 (2001).

2. Yurke, B., Turberfield, A. J., Mills, A. P. Jr., Simmel, F. C., & Neumann, J. L.: A DNA-fueled molecular machine made of DNA, Nature 406, 605-608 (2000).

3. Kelly, T. R., de Silva, H. & Silva, R. A. Unidirectional rotary motion in a molecular system, Nature 401, 150-152 (1999).

4. Mao, C., Sun, W., Shen, Z. & Seeman N. C. A nanomechanical device based on the B-Z transition of DNA, Nature 397, 144-146 (1999).

5. Soong, R. K., Bachand, H. P., Neeves, H. P., Olkhovets, A. G., Craighead, H. G. S & Montemagno, C. D. Powering an inorganic nanodevice with a biomolecular motor, Science 290, 1555-1558 (2000).

6. Jimenez, M. C., Dietrich-Buchecker, C., Sauvage, J.-P. Towards synthetic molecular muscles: construction and stretching of a linar rotaxane dimer, Angrew. Chem. Int. Edn. 39, 3284-3286(2000).

7. Stojanovic, M. N., de Prada, P. & Laundry, D. W. Homogenous Assays Based on Deoxyribozymes, Nucleic Acids Res. 28, 2915-2918(2000).

8. Stojanovic, M. N., de Prada, P. & Laundry, D. W. Catalytic Molecular Beacons, Chembiochem. 2, 411-415(2001).

9. Stojanovic, M. N., de Prada, P. & Laundry, D. W. Fluorescent Sensors Based on Aptamer Self-Assembly, J. Am. Chem. Soc. 122, 11547-11548(2000).

10. Stojanovic, M. N., de Prada, P. & Laundry, D. W. Aptamer-Based Folding Fluorescent Sensor for Cocaine, J. Am. Chem. Soc.123, 4928-4931(2001).

11. Stojanovic, M. N., Mitchell, T. E. & Stefanovich, D Deoxyribozsyme-Based Logic Gates, J. Am. Chem. Soc. accepted for publication, estimated publication date in May 2002.

12. Li, Y. & Breaker, R. R. Deozyribozymes: new players in the ancient game of biocatalysis, Curr. Opin. Struct. Biol. 9(3), 315-323(1999).

13. Breaker, R. R. & Joyce, G. F. A DNA enzyme with Mg²⁺-dependent RNA phosphodiesterase activity, Chem. Biol. 2, 655-660(1995).

14. Santoro, S. W. & Joyce, G. F. A A general purpose RNA-cleaving DNA enzyme, Proc. Natl. Acad. Sci. 94 4262-4266(1997).

15. Li, J. & Lu, Y. J. Am. Chem. Soc. 122, 10466-10477(2000).

16. Guo, Z., Guilfoyle, R. J., Wang, R. & Smith, L. M. Direct fluorescence analysis of genetic polymortphisms by hybridization with oligoncucleotide arrays on glass support, Nucliec Acids Res. 22, 5456-5465(1994).

17. Kumar, A., Larson, O., Parodi, D. & Liang, Z. Silanized nucleic acids: a general platform for DNA immobilization, Nucleic Acids Res. 28, E71(2000).

I claim:

1. A macromolecular assembly comprising a body and at least four catalytic leg units having nucleic acids, the assembly being adapted to travel across a layer of feed oligonucleotide fuel substrate molecules wherein each catalyic leg unit recognizes and binds to a fuel substrate, cleaves the fuel substrate and searches for a new fuel substrate, said leg units alternately binding and cleaving out of phase to keep at least one leg unit bound to a fuel substrate.

2. The macromolecule of claim 1, wherein the leg units have the same nucleic acids.

3. The macromolecule of claim 1, wherein the leg units have different nucleic acids.

4. The macromolecule of claim 1, comprising at least six catalytic leg units.

5. The macromolecule of claim 1, wherein the four leg units are arranged in a tetrahedral relationship.

6. The macromolecule of claim 1, wherein the four leg units are arranged in a rectangular relationship.

7. The macromolecule of claim 1, wherein the leg units are comprised of DNA enzymes.

8. The macromolecule of claim 1, wherein the leg units are comprised of RNA enzymes.

9. The macromolecule of claim 1, wherein the body is comprised of streptavidine.

10. The macromolecule f claim 1, wherein the body is comprised of DNA.

11. The macromolecule of claim 1, wherein the body is comprised of RNA.

12. The macromolecule of claim 1, wherein the leg units include flexible polyethylene glycolspacers.

13. A macromolecular system, comprising the macromolecule of claim 1, and a feed layer having oligonucleotide substrates as fuel molecules.

14. The system of claim 13, wherein the oligonucleotides are made of DNA.

15. The system of claim 13, wherein the oligonucleotides are made of RNA.

16. The system of claim 13, wherein the oligonucleotides are made of a mixture of DNA and RNA.

17. The system of claim 13, wherein the fuel molecules are arranged in a gradient on the substrate.

18. The system of claim 13, wherein the leg units comprise different nucleic acids.
